(19) United States
(12) Patent Application Publication
(10) Pub. No.: US 2005/0076919 A1
(43) Pub. Date: Apr. 14, 2005

(54) Scapula stabilization method and apparatus

(76) Inventor: Joseph Muzzio, Golden, CO (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/681,911

(22) Filed: Oct. 10, 2003

(51) Int. Cl. A61B 19/00

(52) U.S. Cl. 128/869; 128/870

(57) ABSTRACT

A physical therapy device that limits undesirable scapula motion and provides tactile feedback to a patient performing shoulder rehabilitation exercises.

### RELATED APPLICATIONS

None.

### GOVERNMENT INTEREST

None.

### BACKGROUND

1. Field of the Invention

I have invented a new way to stabilize the shoulder or scapula. This is useful during rehabilitation of shoulder injury and for post-surgical patients.

2. Description of Related Art

Physical therapy for shoulder injuries may entail the patient raising their arm(s). When a person raises their arm, however, the person often has a natural tendency to “hike” their shoulder joint, lifting the shoulder joint a bit to ease the work of lifting one's arm. Leonardo da Vinci shows this aptly in one of his drawings, reproduced at FIG. 1. As shown in FIG. 1, raising one's arms laterally raises or “hikes” the shoulder joint above the natural level at rest.

Hiking the shoulder joint, however, interferes with the effectiveness of certain physical therapy exercises. Shoulder hiking is a common, and often frustrating, problem faced by physical therapists when working with post-surgical shoulder patients. Especially when there is inadequate range of motion in the glenohumeral joint, many patients will inadvertently utilize the scapulothoracic joint to achieve motion above the 90 degree frontal and horizontal plane. This in turn causes improper scapulohumoral motion, which should occur in a 2:1 Ratio, as described by Dr. CODMAN.

To prevent this, a physical therapist simply manually applies pressure to the patient's shoulder joint, to prevent shoulder hiking. While this approach is effective, it is also expensive, requiring a highly-trained specialist to spend time holding the patient's shoulder at proper height. Thus, the art needs a cost-effective and clinically effective way to prevent a patient from hiking their shoulder during shoulder exercises.

### BRIEF SUMMARY

I have found a way to accomplish this. My invention is both simple (thus inexpensive to make and easy to use) and effective. It is based on the insight that hiking requires increasing the distance between the patient's hip cage and the top of their shoulder. My invention thus entails preventing an increase in this distance. I accomplish this by placing in contact with the superior aspect of the patent's shoulder, an immobile restraint which physically prevents the patient from increasing the distance between their hip and the top of their shoulder. This prevents shoulder hiking.

### BRIEF DESCRIPTION OF THE FIGURES

FIG. 1 illustrates how raising one's arm can result in shoulder hiking.

FIG. 2 shows the distance d between the hips and the superior aspect of the shoulder.

FIG. 3 is an isometric perspective of my currently-preferred version or embodiment of my invention.

FIG. 4 provides a front view of my currently-preferred version.

FIG. 5 provides a rear view of my currently-preferred version.

FIG. 6 provides a right side view of my currently-preferred version.

FIG. 7 provides a left side view of my currently-preferred version.

FIG. 8 provides a top view of my currently-preferred version.

FIG. 9 provides a bottom view of my currently-preferred version.

FIG. 10 illustrates an alternative embodiment of my invention.

FIG. 11 illustrates an alternative embodiment of my invention.

FIG. 12 illustrates an alternative embodiment of my invention.

FIG. 13 illustrates an alternative embodiment of my invention.

### DETAILED DESCRIPTION

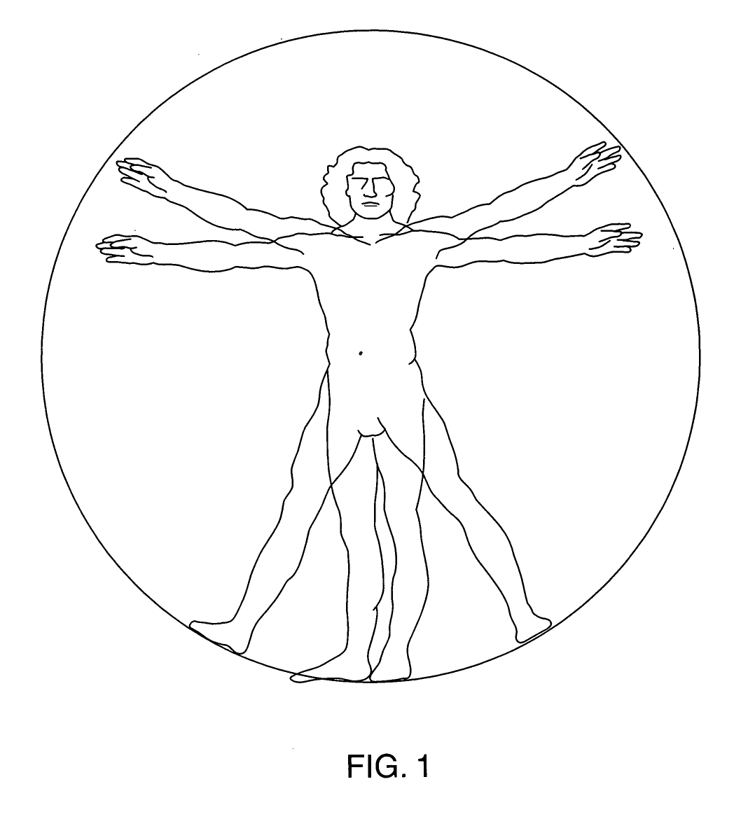

When a person raises their arm, the person often has a natural tendency to “hike” their shoulder joint, lifting the shoulder joint a bit to ease the work of lifting one's arm. This is illustrated at FIG. 1. As shown in FIG. 1, raising one's arms laterally can raise or “hike” the superior aspect of the shoulder joint.

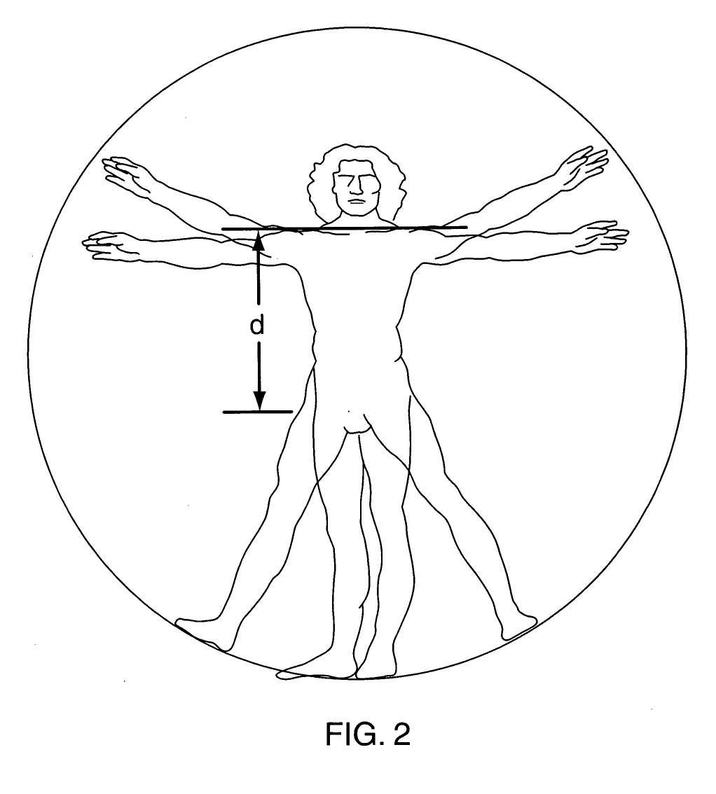

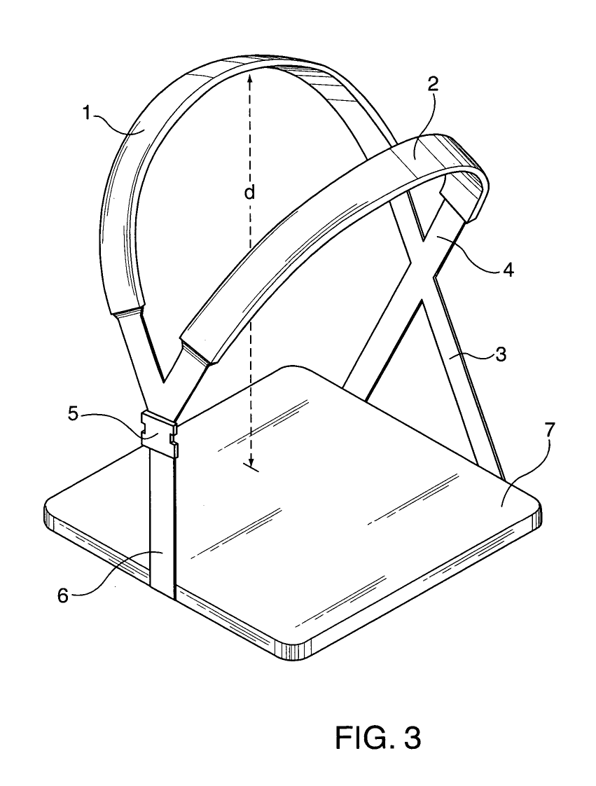

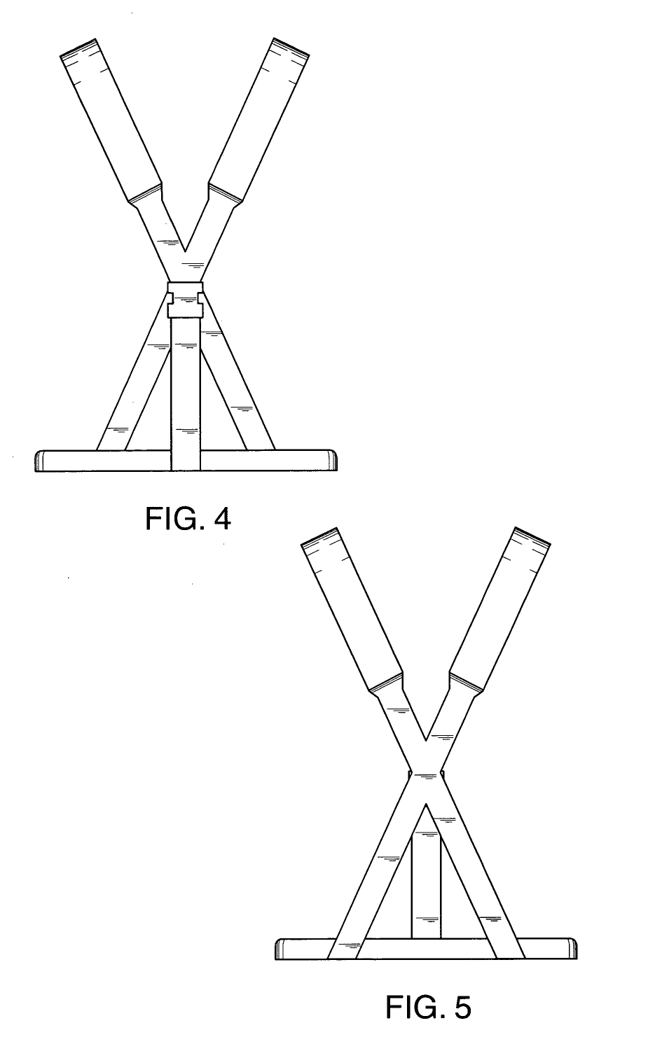

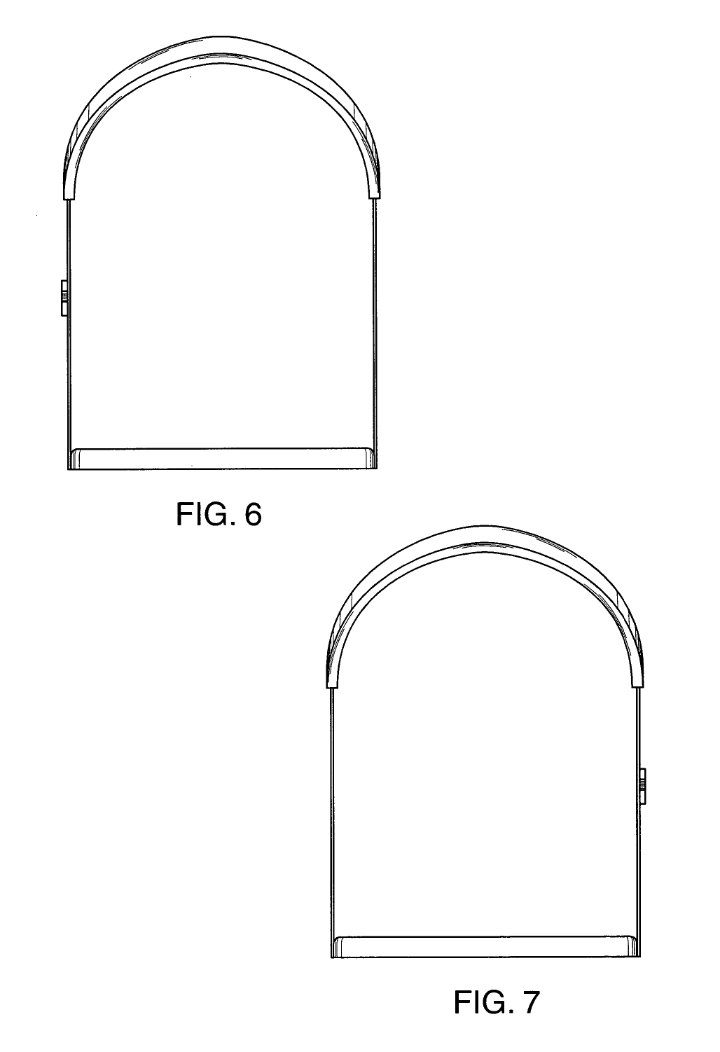

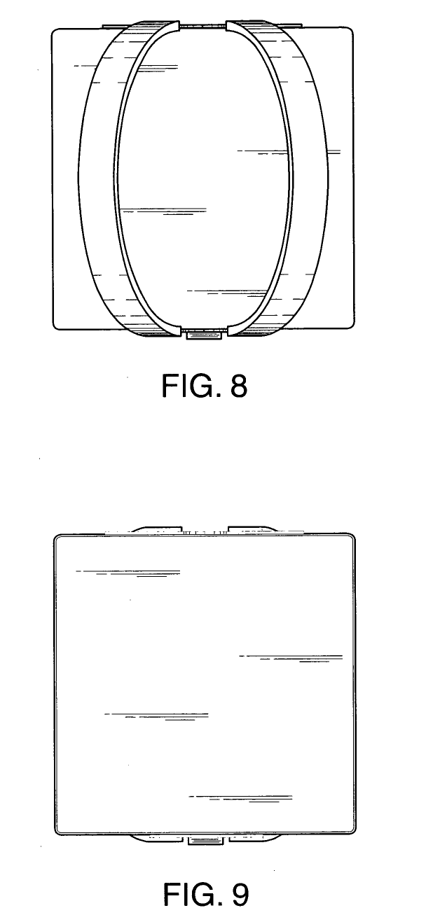

My invention is a physical therapy device that limits undesirable scapula motion. This is shown in FIG. 2. In FIG. 2, the superior aspect of the shoulder is kept below a certain pre-defined position. I prefer to define this position as a defined distance d from the patient's hips.

My invention can provide tactile feedback to a patient performing shoulder rehabilitation exercises. My invention limits the motion of the scapula without requiring a physical therapist, ensuring that the patient can independently execute proper technique. While my invention does not eliminate the need for formal physical therapy, my invention eliminates the need for a physical therapist to be present at all times to ensure proper shoulder stabilization. My invention enjoys a quite simple design, which enables the patient to independently use the device after initial instruction.

My invention entails placing the patient in a restrained position, evaluating the patient's desired shoulder position, and then placing an restraint above the shoulder and in contact with the shoulder, and immobilizing the restraint vis the patient's hips. The restraint then prevents the shoulder from exceeding the desired position.

By placing the patient in a “restrained position” I mean that the patient is in a position where the hips are restrained from moving in relation to the shoulder restraint. This may be done in a variety of ways. The easiest way, and the way I prefer, is to have the patient sit down in a chair, using good posture. By sitting down, the patient's hips are fixed in space. Alternatively, the patient may lie on a table, or even stand upright. If the patient stands upright, however, the patient should lock their knees, or lean against a wall, or do something else to prevent their hips from moving.

Once the patient is in position, a shoulder restraint is placed at the desired position in contact with the patient's shoulder. I define this position as the distance d between the patient's hips and outside of shoulder. The shoulder restraint can be made of a belt, a padded bar, or any other means to provide a relatively immobile object which the patient's shoulder cannot move. The shoulder restraint is then fixed in position relative to the patient's hips.

This may be done in a variety of ways. For example, as of this writing I prefer to stabilize the shoulder or scapula with two padded shoulder straps, attached onto a seat. I show my preferred embodiment in FIG. 3. In FIG. 3, a first shoulder pad [1] and a second shoulder pad [2] are fixed to a first belt [3] and a second belt [4] respectively. A terminal end of each belt [3, 4] are joined at a clip [5]. Also attached to the clip [5] is an end of a third belt [6]. The third belt [6] is also attached, at its opposite end, to the front edge of a seat [7]. The clip [5] provides an adjustable connection so that the length of the third belt [6] may be adjusted. This adjusts the length of the third belt [6]. In so doing, then, one can adjust the distance d between the shoulder pads [1, 2] and the seat [7]. Adjusting distance d accommodates patients of various heights.

The first belt [3] and the second belt [4] are, at their opposite terminal ends, attached to the rear edge of the seat [7].

In use, the seat [7] is placed on a standard chair. The clip [5] is unfastened, and the patient sits down on the seat [7], facing the front edge of the seat, using good posture. The first and second straps [3, 4] are placed along the patient's back, and the patient puts his or her head between the shoulder pads [1, 2], so that the shoulder pads [1, 2] rest on the superior aspect of the patient's shoulders. The two straps are then brought across the top of the patients' shoulders and down the patient's chest. The first and second straps [1, 2] are then connected with the clip [5] to the third strap [6]. The length of the third strap [6] is adjusted so that the shoulder pads [1, 2] meet the superior aspect of the patient's shoulders snugly but comfortably.

So adjusted, the patient can now begin to do their shoulder exercises, lifting their arm(s) either to the front, to the side, etc. . . . If and when the patient begins to hike their shoulder, the superior aspect of their shoulders will push against the shoulder pads [1 or 2]. The shoulder pads, immobilized by the straps [3, 4, 6], prevent the shoulder from hiking.

After the patient finishes their exercises, the patient unfastens the clip [5] and can then step out of the device. The entire device may then be removed from the chair, packed up and stored away.

My device may be made to fit onto a standard chair, as shown in FIG. 3. Alternatively, the device may be made to itself be a chair. That is to say, the seat [7] can have chair legs attached to the bottom of it. This may prove attractive where a standard chair is unsafe or unsuited, for example with impaired patients at risk of falling off a standard chair.

Alternatively, the device can be provided as a kit comprising straps [3, 4, 6] with connectors on the ends, which are connectable to a standard chair. This version is less expensive and easier to ship, albeit somewhat less convenient to install for use. Similarly, the device can be provided as a kit comprising straps [3, 4, 6] long enough to reach across a standing patient's shoulders to the floor, whereby the patient can do their exercises while standing. In these various embodiments, the common thread is that the device is able to define a fixed distance d between the patient's shoulders, and the patient's hips, so that the patient's shoulder position vis the hip position is preserved and shoulder hiking prevented.

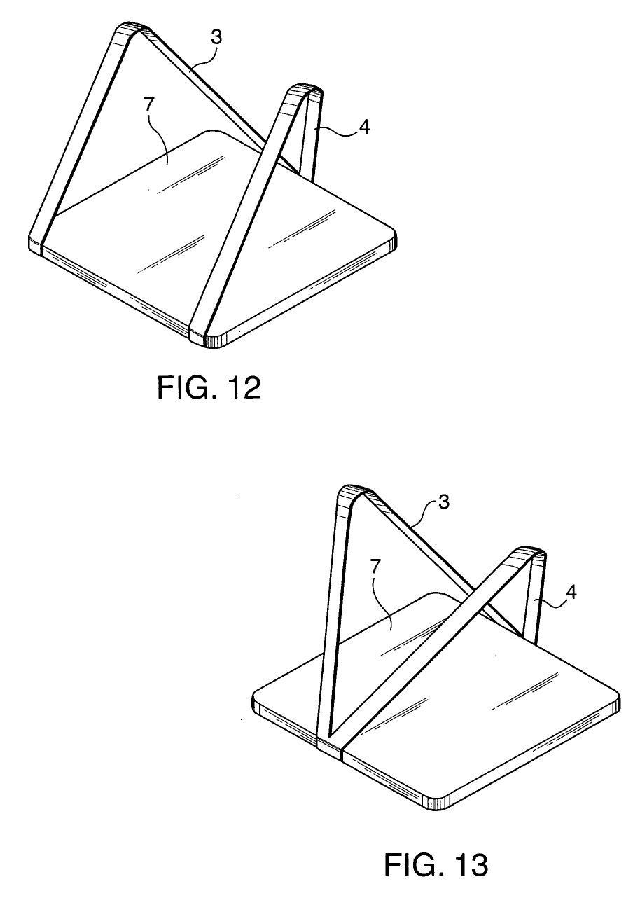

The precise means for restraining shoulder hiking is a design choice. As of this writing, I prefer to restrain shoulder hiking by use of straps which define a fixed distance from the patient's hips (or feet, etc. . . . ). The precise array of straps is simply a design choice. Thus, for example, one may have the first strap [3] and the second strap [4] continue from the back of the seat [7] all the way to the front face of the seat, as shown in FIGS. 11-13. Similarly, it is conceivable that one may make a version of my invention with only one strap, for a patient with only one shoulder injured, for example, or for a patient to do their exercises only one shoulder/side at a time.

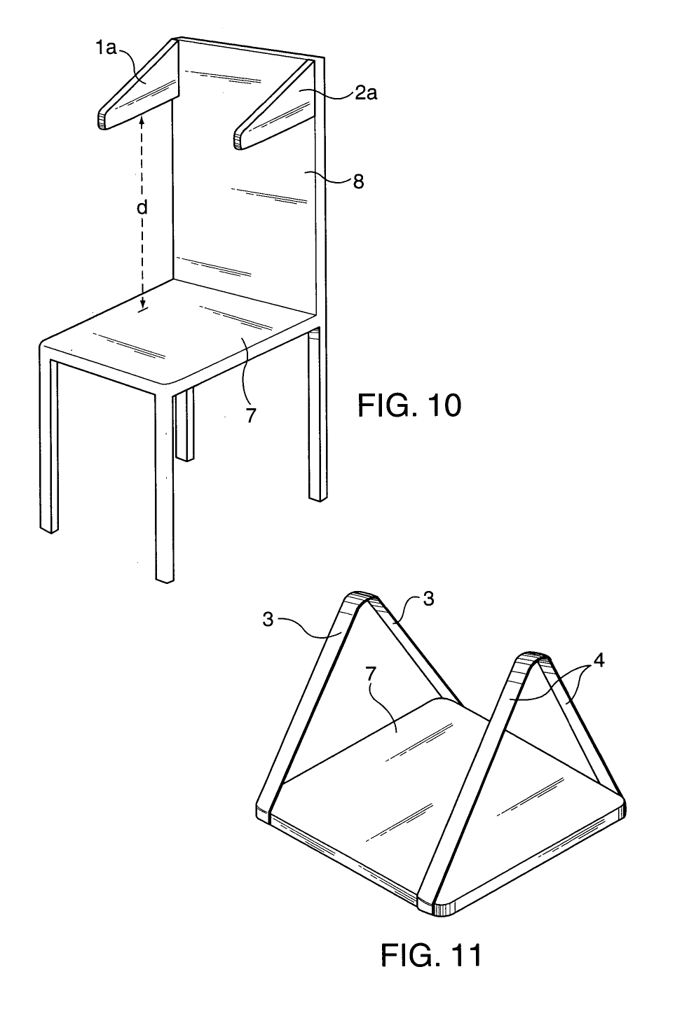

As an alternative to straps, one may provide alternative means to prevent hiking. For example, FIG. 10 shows a standard “ladder-back” or high-back chair with a ladder back [8]. The ladder-back [8] bears posts [1a, 2a] which are perpendicular to the high back [8]. The posts [1a, 2a] are adjustable to define varying distances d above the seat [7]. When the patient sits in the chair seat [7], the posts [1a, 2a] meet the superior aspect the patient's shoulders, and thus prevent shoulder hiking in the same way, to produce the same result, as do the shoulder pads [1, 2] shown in FIG. 3.

In using my device, some patients may frustrate its function by using improper form. For example, after the hip-shoulder distance d is set, the patient may slouch down in the chair. This can defeat the function of the device, where the hike-prevention means is set at a static distance (such as, for example, with a version where the hike prevention means is positioned using non-stretch straps. The function of my device can easily be preserved, however, by using a lumbar roll to support the patient's back, perhaps in conjunction with a full-length mirror for “visual cueing” (enabling the patient to see their own actions and correct them as required). In so doing, the patient avoids slouching.

My invention may be used with a wall pulley unit during passive-range of motion exercises. In this, my device prevents hiking of the shoulder, which leads to improper scapulohumeral rhythm. My device can also be used in the later stages of shoulder rehabilitation, with active or resistive exercises, to also prevent hiking of the shoulder.

After reading my invention here, a variety of variations will become readily apparent to one of skill in the art. Thus, I intend that the coverage of this patent be defined not by any of the specific versions or preferred embodiments I discuss here; to the contrary, I intend the scope of this patent to be defined by the various patent claims.

In the claims, I use the singular (e.g., the terms “a” and “one”) to include the plural (i.e., to mean, “at least one, but possible more than one”).

The preamble is part of the claim. It is necessary to give meaning to the claim. As such, this language is not simply an intended use, but defines the subject matter of the claimed invention. The claim preamble limits the claim because the preamble “is ‘necessary to give life, meaning, and vitality’ to the claim.” See Eaton Corp. v. Rockwell Int'l Corp., slip op. 01-1633 (Fed. Cir., 27 Mar. 2003) (a “claim preamble has the import that the claim as a whole suggests for it. In other words, when the claim drafter chooses to use both the preamble and the body to define the subject matter of the claimed invention, the invention so defined, and not some other, is the one the patent protects”).

1. A physical therapy apparatus to prevent shoulder hiking, comprising:
A) a shoulder restraint,
B) said shoulder restraint connected to a connector,
C) said connector connectable to a hip stabilizer,
D) said connector defining a fixed distance between said shoulder restraint and said hip stabilizer.

2. The apparatus of claim 1, wherein said hip stabilizer comprises a seat.

3. The apparatus of claim 1, wherein said shoulder restraint comprises a strap.

4. The apparatus of claim 3, wherein said connector is connected to said hip stabilizer, and wherein said hip stabilizer comprises a seat.

5. The apparatus of claim 3, wherein said hip stabilizer comprises a chair.

6. A method to prevent a patient from hiking their shoulder while performing physical therapy exercises, the method comprising:
A) determining the desired position of said patient's shoulder,
B) placing the patient's shoulder at said desired position, and
C) placing in contact with said patient's shoulder a shoulder restraint, said shoulder restraint immobile relative to said shoulder,
whereby said shoulder restraint prevents said patient's shoulder from hiking beyond said desired position.

7. The method of claim 6, wherein said shoulder restraint comprises a strap.

8. The method of claim 7, wherein said shoulder restraint is connected to a connector, and said connector is connected to a hip stabilizer, whereby said shoulder restraint is maintained relatively immobile.

9. The method of claim 8, wherein said hip stabilizer comprises a seat.

10. The method of claim 8, wherein said hip stabilizer comprises a chair.
